(19) United States
(12) Patent Application Publication
(10) Pub. No.: US 2008/0234325 A1
(43) Pub. Date: Sep. 25, 2008

(54) Novel Methods Using Pyridine Derivatives

(75) Inventors: John Ieni, Glen Ridge, NJ (US); Jay Barth, Teaneck, NJ (US); Robert M. Niecestro, Pocono Pines, PA (US)

Correspondence Address: Lahive & Cockfield, LLP/EISAI;Floor 30, Suite 3000, One Post Office Square, Boston, MA, 02109, US

(73) Assignee: Eisai Co., Ltd.

(21) Appl. No.: 10/311,833

(22) PCT Filed: Jun. 19, 2001
(86) PCT No.: PCT/US01/19420

§ 371 Date: Sep. 19, 2005

Related U.S. Application Data
(60) Provisional application No. 60/212,637, filed on Jun. 19, 2000.
(60) Provisional application No. 60/243,278, filed on Oct. 26, 2000.

(51) Int. Cl. A61K 31/4439; A61P 1/00

(52) U.S. Cl. 514/338

(57) ABSTRACT

The invention provides methods of treating and preventing asthma, laryngitis, symptomatic gastroesophageal reflux disease, pregnancy-induced gastroesophageal reflux disease, noncardiac chest pains, coughing, apnea, dyspepsia, inflammatory bowel disease, irritable bowel syndrome, gastritis, stress ulcers, bleeding peptic ulcers, acute gastrointestinal bleeding, infectious enteritis, collagenous colitis, lymphocytic colitis, chronic diarrhea in immunocompromised patients, esophageal ulcers in immunocompromised patients, idiopathic gastric acid hypersecretion, gastroparesis, gastrointestinal motility disorders, Zollinger-Ellison syndrome, short bowel syndrome, emesis, regurgitation, early satiety, chronic sore throat, abdominal pain, abdominal bloating, nausea, sour stomach, diarrhea, constipation, bacterial infections, refractory ulcers, gastrointestinal disorders induced by NSAIDs, Barrett's esophagus, gastrointestinal disorders caused by steroids, gastrointestinal disorders induced by cholinergic compounds, and fungal or viral-induced ulcers in the gastrointestinal tract by administering a therapeutically effective amount of at least one of the pyridine derivatives of the invention to a patient in need thereof. The invention also provides on demand relief of symptoms associated with gastroesophageal reflux disease (GERD), and provides relief from symptoms caused by the consumption of excessive amounts of food and/or alcohol by administering a therapeutically effective amount of at least one of the pyridine derivatives of the invention to a patient in need thereof. The invention also provides methods for treating parasitic infections, such as malaria, by administering a therapeutically effective amount of at least one of the pyridine derivatives of the invention to a patient in need thereof. In preferred embodiments, the pyridine derivative of the invention is rabeprazole, a pharmaceutically acceptable salt thereof, or a stereoisomer thereof.

### RELATED APPLICATIONS

The present application claims priority to U.S. Provisional Application No. 60/243,278 filed Oct. 26, 2000, and U.S. Provisional Application No. 60/212,637 filed Jun. 19, 2000.

### FIELD OF THE INVENTION

The invention provides safe and effective methods for treating and preventing gastrointestinal diseases and other disorders in patients by administering one or more pyridine derivatives. In preferred embodiments, the pyridine derivative is rabeprazole, a pharmaceutically acceptable salt thereof, or a stereoisomer thereof. In other preferred embodiments, the pyridine derivative is rabeprazole sodium or ACIPHEX®.

### BACKGROUND OF THE INVENTION

Duodenal and gastric ulcers, known collectively as peptic ulcers, are localized erosions of the mucous membrane of the duodenum and stomach, respectively, which expose the underlying layers of the gut wall to the acid secretions of the stomach and to the proteolytic enzyme pepsin. They are believed to be caused by an imbalance between offensive factors, such as acid or pepsin, and defensive factors, such as resistance of the mucous membrane. Peptic ulceration is a common disease of the gastrointestinal tract and it is estimated that approximately 10 to 20% of the adult male population will experience peptic ulceration at some time in their lives.

Proton pump inhibitors, such as ACIPHEX® (Eisai Inc., Teaneck, N.J.), have proven to be successful in treating peptic ulcers. ACIPHEX® is described in U.S. Pat. No. 5,045,552, the disclosure of which is incorporated herein by reference in its entirety.

There is a need in the art for new and improved treatments for other gastrointestinal diseases and disorders. The invention is directed to these, as well as other, important ends.

### SUMMARY OF THE INVENTION

The invention provides methods of treating and preventing asthma in patients by administering a therapeutically effective amount of at least one of the pyridine derivatives of the invention.

The invention provides novel methods for treating and preventing laryngitis in patients by administering a therapeutically effective amount of at least one of the pyridine derivatives of the invention.

The invention provides novel methods for treating and preventing symptomatic gastroesophageal reflux disease in patients by administering a therapeutically effective amount of at least one of the pyridine derivatives of the invention.

The invention provides novel methods for treating and preventing pregnancy-induced gastroesophageal reflux disease in patients by administering a therapeutically effective amount of at least one of the pyridine derivatives of the invention.

The invention provides novel methods for treating and preventing noncardiac chest pains in patients by administering a therapeutically effective amount of at least one of the pyridine derivatives of the invention.

The invention provides novel methods for treating and preventing coughing in patients by administering a therapeutically effective amount of at least one of the pyridine derivatives of the invention. The invention also provides novel methods for treating and preventing bronchitis in patients by administering a therapeutically effective amount of at least one of the pyridine derivatives of the invention.

The invention provides novel methods for treating and preventing apnea in patients by administering a therapeutically effective amount of at least one of the pyridine derivatives of the invention.

The invention provides novel methods for treating and preventing dyspepsia in patients by administering a therapeutically effective amount of at least one of the pyridine derivatives of the invention.

The invention provides novel methods for treating and preventing inflammatory bowel disease, including ulcerative colitis and Crohn's disease, in patients by administering a therapeutically effective amount of at least one of the pyridine derivatives of the invention.

The invention provides novel methods for treating and preventing irritable bowel syndrome in patients by administering a therapeutically effective amount of at least one of the pyridine derivatives of the invention.

The invention provides novel methods for treating and preventing gastritis in patients by administering a therapeutically effective amount of at least one of the pyridine derivatives of the invention.

The invention provides novel methods for treating and preventing gastroesophageal reflux disease and peptic ulcers in infants and children by administering a therapeutically effective amount of at least one of the pyridine derivatives of the invention.

The invention provides novel methods for treating and preventing stress ulcers in patients by administering a therapeutically effective amount of at least one of the pyridine derivatives of the invention.

The invention provides novel methods for treating and preventing bleeding peptic ulcers and for treating and preventing acute gastrointestinal bleeding in patients by administering a therapeutically effective amount of at least one of the pyridine derivatives of the invention.

The invention provides novel methods for treating and preventing infectious enteritis in patients by administering a therapeutically effective amount of at least one of the pyridine derivatives of the invention.

The invention provides novel methods for treating and preventing collagenous colitis and lymphocytic colitis in patients by administering a therapeutically effective amount of at least one of the pyridine derivatives of the invention.

The invention provides novel methods for treating and preventing chronic diarrhea in immunocompromised patients, including patients with transplants, AIDS or HIV infection by administering a therapeutically effective amount of at least one of the pyridine derivatives of the invention.

The invention provides novel methods for treating and preventing ulcers in the esophagus in immunocompromised patients, including patients with transplants, AIDS or HIV infection by administering a therapeutically effective amount of at least one of the pyridine derivatives of the invention.

The invention provides novel methods for treating and preventing idiopathic gastric acid hypersecretion in patients by administering at least one of the pyridine derivatives of the invention to a patient.

The invention provides novel methods for treating and preventing gastroparesis in patients by administering a therapeutically effective amount of at least one of the pyridine derivatives of the invention.

The invention provides novel methods for treating and preventing gastrointestinal motility disorders in patients by administering at least one of the pyridine derivatives of the invention.

The invention provides novel methods for treating and preventing Zollinger-Ellison syndrome in patients by administering a therapeutically effective amount of at least one of the pyridine derivatives of the invention.

The invention provides novel methods for treating short bowel syndrome in patients by administering a therapeutically effective amount of at least one of the pyridine derivatives of the invention.

The invention provides novel methods for treating and preventing emesis in patients by administering a therapeutically effective amount of at least one of the pyridine derivatives of the invention.

The invention provides novel methods for treating and preventing regurgitation in patients by administering a therapeutically effective amount of at least one of the pyridine derivatives of the invention.

The invention provides novel methods for treating and preventing early satiety in patients by administering a therapeutically effective amount of at least one of the pyridine derivatives of the invention.

The invention provides novel methods for treating and preventing chronic sore throat in patients by administering a therapeutically effective amount of at least one of the pyridine derivatives of the invention.

The invention provides novel methods for treating and preventing abdominal pain in patients by administering a therapeutically effective amount of at least one of the pyridine derivatives of the invention.

The invention provides novel methods for treating and preventing abdominal bloating in patients by administering a therapeutically effective amount of at least one of the pyridine derivatives of the invention.

The invention provides novel methods for treating and preventing nausea in patients by administering a therapeutically effective amount of at least one of the pyridine derivatives of the invention.

The invention provides novel methods for treating and preventing sour stomach in patients by administering a therapeutically effective amount of at least one of the pyridine derivatives of the invention.

The invention provides novel methods for treating and preventing diarrhea in patients by administering a therapeutically effective amount of at least one of the pyridine derivatives of the invention.

The invention provides novel methods for treating and preventing constipation in patients by administering a therapeutically effective amount of at least one of the pyridine derivatives of the invention.

The invention provides novel methods for treating and preventing bacterial infections in patients by administering a therapeutically effective amount of at least one of the pyridine derivatives of the invention.

The invention provides novel methods for treating and preventing refractory ulcers in patients by administering a therapeutically effective amount of at least one of the pyridine derivatives of the invention.

The invention provides on demand relief of symptoms associated with gastroesophageal reflux disease (GERD) in patients by administering a therapeutically effective amount of at least one of the pyridine derivatives of the invention.

The invention provides relief from symptoms caused by the consumption of excessive amounts of food and/or alcohol in patients by administering a therapeutically effective amount of at least one of the pyridine derivatives of the invention.

The invention provides novel methods for treating and preventing Barrett's esophagus in patients by administering a therapeutically effective amount of at least one of the pyridine derivatives of the invention.

The invention provides novel methods for treating and preventing gastrointestinal disorders induced by NSAIDs in patients by administering a therapeutically effective amount of at least one of the pyridine derivatives of the invention.

The invention provides novel methods for treating and preventing gastrointestinal disorders caused by steroids in patients by administering a therapeutically effective amount of at least one of the pyridine derivatives of the invention.

The invention also provides novel methods for treating and preventing gastrointestinal disorders caused by cholinergic compounds in patients by administering a therapeutically effective amount of at least one of the pyridine derivatives of the invention.

The invention provides novel methods for treating and preventing fungal-induced or viral-induced ulcers in the gastrointestinal tract in patients by administering a therapeutically effective amount of at least one of the pyridine derivatives of the invention.

The invention provides a novel diagnostic tool for suppressing gastric acid.

The invention provides novel methods for treating parasitic infections, such as malaria, in patients and for modulating the growth of parasites by administering an effective amount of at least one of the pyridine derivatives of the invention.

The invention is described in more detail below.

### DETAILED DESCRIPTION OF THE INVENTION

“Patient” includes animals, preferably mammals, more preferably humans. “Patient” includes infants, children and adults, and includes males and females.

“Gastroesophageal Reflux Disease” or “GERD” refers to a clinical syndrome involving the reflux of gastric contents into the esophagus, and is generally characterized by one or more symptoms of heartburn, coughing, wheezing, hoarseness, regurgitation, epigastric pain, dysphagia, and chest pain.

The invention provides methods of treating and preventing asthma in infants, children and adults by administering a therapeutically effective amount of at least one of the pyridine derivatives of the invention. GERD is common in patients with asthma, although no causal relationship between the two diseases has been proven. In other embodiments, the invention provides methods for treating and preventing asthma in patients who are also diagnosed with GERD.

The invention provides methods for treating and preventing laryngitis by administering a therapeutically effective amount of at least one of the pyridine derivatives of the invention to a patient in need thereof. In other embodiments, the invention provides methods for treating and preventing laryngitis in patients who are also diagnosed with GERD. The invention also provides methods for preventing and treating laryngeal carcinoma by administering a therapeutically effective amount of at least one of the pyridine derivatives of the invention.

The invention provides methods for treating and preventing Barrett's esophagus by administering a therapeutically effective amount of at least one of the pyridine derivatives of the invention to a patient in need thereof. Barrett's esophagus is a condition in which the stratified squamous epithelium of the esophagus is replaced by a columnar epithelium with malignant potention.

The invention provides methods for treating and preventing symptomatic gastroesophageal reflux disease (GERD) by administering a therapeutically effective amount of at least one of the pyridine derivatives of the invention to a patient in need thereof. Symptomatic GERD is characterized by the presence of symptoms of GERD, most commonly heartburn, which are related to the reflux of gastric contents into the esophagus. Symptomatic GERD is distinguished from GERD (or erosive GERD) by the absence of erosions in the esophageal mucosa.

The invention provides methods for preventing and treating pregnancy-induced gastroesophageal reflux disease by administering a therapeutically effective amount of at least one of the pyridine derivatives of the invention to a pregnant female patient in need thereof.

The invention provides methods for treating and preventing noncardiac chest pains by administering a therapeutically effective amount of at least one of the pyridine derivatives of the invention to a patient in need thereof. In other embodiments, the invention provides methods for treating and preventing noncardiac chest pains in patients who are also diagnosed with GERD.

The invention provides methods for treating and preventing coughing, preferably chronic coughing, by administering a therapeutically effective amount of at least one of the pyridine derivatives of the invention to a patient in need thereof. In other embodiments, the invention provides methods for treating and preventing coughing in patients who are also diagnosed with GERD. The invention also provides methods for treating and preventing bronchitis.

The invention provides methods for treating and preventing apnea in patients by administering a therapeutically effective amount of at least one of the pyridine derivatives of the invention. The patient can be an infant, child or adult. In one embodiment, the patient is preferably an infant. The term “infant” includes neonates.

The invention provides methods for treating and preventing dyspepsia, preferably non-ulcer or functional dyspepsia, by administering a therapeutically effective amount of at least one of the pyridine derivatives of the invention to a patient in need thereof. Dyspepsia refers to upper abdominal pain or discomfort and can also include symptoms of nausea, early satiety and bloating. Dyspepsia can be episodic or chronic.

The invention provides methods for treating and preventing inflammatory bowel disease by administering a therapeutically effective amount of at least one of the pyridine derivatives of the invention to a patient in need thereof. Inflammatory bowel disease refers to chronic inflammatory disorders involving the gastrointestinal tract, and is characterized by symptoms of diarrhea, bloody diarrhea, perianal sepsis, and/or abdominal pain. Inflammatory bowel disease includes ulcerative colitis and Crohn's disease. Ulcerative colitis is an inflammatory reaction primarily involving the colonic mucosa, and is characterized by symptoms of bloody diarrhea, abdominal pain, fever, and/or weight loss. Crohn's disease is a chronic inflammation extending through all layers of the intestinal wall and involving the mesentery and regional lymph nodes and can involve the small bowel and/or colon. Crohn's disease is characterized by symptoms of fever, abdominal pain, diarrhea often without blood, fatigue, and/or weight loss.

The invention provides methods for treating and preventing irritable bowel syndrome by administering a therapeutically effective amount of at least one of the pyridine derivatives of the invention to a patient in need thereof. Irritable bowel syndrome is a common gastrointestinal disease characterized by three clinical variants: (i) chronic abdominal pain and constipation, (ii) chronic intermittent diarrhea, often without pain, and (iii) alternating constipation and diarrhea, with or without abdominal pain.

The invention provides methods for treating and preventing gastritis by administering a therapeutically effective amount of at least one of the pyridine derivatives of the invention to a patient in need thereof. “Gastritis” refers to inflammation of the gastric mucosa. The term “gastritis” includes acute gastritis and chronic gastritis.

The invention provides methods for treating and preventing gastroesophageal reflux disease and peptic (gastric and duodenal) ulcers in infants and children by administering a therapeutically effective amount of at least one of the pyridine derivatives of the invention to a patient who is an infant or child. The term “infants” includes neonates, and the term “children” includes adolescents.

The invention provides methods for treating and preventing stress ulcers by administering a therapeutically effective amount of at least one of the pyridine derivatives of the invention to a patient in need thereof. Stress ulcers are clinically distinct from peptic ulcers. Patients with stress ulcers often have multiple lesions in the acid-secreting portion of the stomach, in the antrum and/or the duodenum, and the lesions may be bleeding. Stress ulcers may be present in patients with severe injuries, burns, infections and/or shock.

The invention provides methods for treating and preventing bleeding peptic ulcers and for treating and preventing acute gastrointestinal bleeding by administering a therapeutically effective amount of at least one of the pyridine derivatives of the invention to a patient in need thereof. The bleeding peptic ulcers may be duodenal or gastric, or stomal ulcers. Gastrointestinal bleeding is a general term referring to bleeding from anywhere in the gastrointestinal tract. Many cases are due to peptic ulcers, but other causes are esophageal and intestinal bleeding. Bleeding is a potential complication of peptic ulcers, and is often associated with more severe ulcers.

The invention provides methods for treating and preventing infectious enteritis by administering a therapeutically effective amount of at least one of the pyridine derivatives of the invention to a patient in need thereof. Infectious enteritis refers to pathogen-induced diarrhea. Infectious enteritis can be caused by an infection from, for example, Campylobacter species, Shigella species, Yersinia species, such as Yersinia enterocolitica, Cryptosporidium species, Giardia species, such as Giardia lamblia, Salmonella species, Pseudomonas species, such as Pseudomonas aeruginosa, and the like.

The invention provides methods for treating and preventing collagenous colitis and lymphocytic colitis by administering a therapeutically effective amount of at least one of the pyridine derivatives of the invention to a patient in need thereof. Both conditions are characterized by signs of mucosal inflammation and symptoms of chronic watery diarrhea.

The invention provides methods for treating and preventing chronic diarrhea and esophageal ulcers in immunocompromised patients, including patients with transplants, AIDS or HIV infection, by administering a therapeutically effective amount of at least one of the pyridine derivatives of the invention.

The invention provides methods for treating and preventing idiopathic gastric acid hypersecretion by administering at least one of the pyridine derivatives of the invention to a patient in need thereof.

The invention provides methods for treating and preventing gastroparesis by administering a therapeutically effective amount of at least one of the pyridine derivatives of the invention to a patient in need thereof. Gastroparesis is delayed gastric emptying of either solids or liquids, and is accompanied by symptoms of postprandial nausea, epigastric pain/burning, bloating, early satiety, excessive eructation, anorexia and vomiting.

The invention provides methods for treating and preventing gastrointestinal motility disorders by administering at least one of the pyridine derivatives of the invention to a patient in need thereof.

The invention provides methods for treating and preventing Zollinger-Ellison syndrome by administering a therapeutically effective amount of at least one of the pyridine derivatives of the invention to a patient in need thereof. Zollinger-Ellison syndrome refers to ulcer disease of the upper gastrointestinal tract, marked increases in gastric acid secretion, and/or nonbeta islet cell tumors of the pancreas.

The invention provides methods for treating short bowel syndrome by administering a therapeutically effective amount of at least one of the pyridine derivatives of the invention to a patient in need thereof.

The invention provides methods for treating and preventing emesis in infants, children and adults by administering a therapeutically effective amount of at least one of the pyridine derivatives of the invention. In other embodiments, the invention provides methods for treating and preventing emesis in patients who are also diagnosed with GERD. In still other embodiments, the invention provides methods for treating and preventing emesis induced by chemotherapeutic agents.

The invention provides methods for treating and preventing regurgitation, early satiety, chronic sore throat, abdominal pain, abdominal bloating, nausea, sour stomach, diarrhea or constipation by administering a therapeutically effective amount of at least one of the pyridine derivatives of the invention to a patient in need thereof.

The invention provides methods for treating and preventing gastrointestinal bacterial infections by administering a therapeutically effective amount of at least one of the pyridine derivatives of the invention to a patient in need thereof. In preferred embodiments, the gastrointestinal bacterial infection is caused by Helicobacter pylori.

The invention provides methods for treating and preventing refractory ulcers by administering a therapeutically effective amount of at least one of the pyridine derivatives of the invention to a patient in need thereof. The ulcers can be peptic ulcers (e.g., gastric ulcers and duodenal ulcers), and can be present in infants, children or adults. Refractory ulcers are generally defined as ulcers that fail to completely heal after daily treatment with 1 gram of cimetidine for three months.

The invention provides on demand relief of symptoms associated with gastroesophageal reflux disease (GERD). Indications for the treatment of GERD with pyridine derivatives, such as rabeprazole or stereoisomers thereof, require daily administration for four to eight weeks. It has been unexpectedly discovered that pyridine derivatives, such as rabeprazole or stereoisomers thereof, can be administered on a one-time basis to treat occasional symptoms of GERD. For example, rabeprazole or stereoisomers thereof can be administered before, during or after a meal to provide relief from GERD symptoms caused by the meal.

The invention provides relief from symptoms caused by the consumption of excessive amounts of food and/or alcohol by administering a therapeutically effective amount of at least one of the pyridine derivatives of the invention to a patient in need thereof. The symptoms can be one or more of abdominal bloating, abdominal pain, regurgitation, emesis, nausea, sour stomach, and the like.

The invention provides methods for treating and preventing gastrointestinal disorders (e.g., peptic ulcers) induced by NSAIDs (non-steroidal antiinflammatory drugs) by administering a therapeutically effective amount of at least one of the pyridine derivatives of the invention to a patient in need thereof. All NSAIDs have the potential to cause damage to the gastrointestinal tract, and have been associated with inducing peptic ulcers (e.g., gastric and duodenal ulcers) and gastrointestinal bleeding. NSAIDs cause gastrointestinal damage by two mechanisms: (1) a topical effect that is pH and pKa related, and (2) a systemic effect mediated by cyclooxygenase (COX) inhibition with a reduction in prostaglandin synthesis. Administering one or more of the pyridine derivatives of the invention can heal gastrointestinal ulcers caused by NSAIDs.

The invention provides methods for treating and preventing gastrointestinal disorders (e.g., peptic ulcers) caused by steroids by administering a therapeutically effective amount of at least one of the pyridine derivatives of the invention to a patient in need thereof. There is a well known correlation between steroid therapy and peptic ulcer disease. Messer et al, N. Engl. J. Med., 309(1):21-24 (1983); Wolf et al, J. Pediatr. Gastroenterol. Nutr., 12(2):269-271 (1991). In other embodiments, the invention provides methods for treating peptic ulcers (e.g., gastric or duodenal) induced by corticosteroids.

The invention also provides methods for treating and preventing gastrointestinal disorders (e.g., peptic ulcers) caused by cholinergic compounds (e.g., bethanecol, metoclopramide and the like) by administering a therapeutically effective amount of at least one of the pyridine derivatives of the invention to a patient in need thereof.

The invention also provides novel methods for treating fungal-induced or viral-induced ulcers in the gastrointestinal tract by administering a therapeutically effective amount of at least one of the pyridine derivatives of the invention to a patient in need thereof.

The invention provides a novel diagnostic tool for suppressing gastric acid. For example, the pyridine derivatives of the invention could be used in the diagnosis of GERD or non-cardiac chest pains.

The invention also provides novel methods for treating parasitic infections by administering a therapeutically effective amount of at least one of the pyridine derivatives of the invention to a patient in need thereof. The parasitic infection is preferably caused by a protozoan parasite, more preferably by Plasmodium falciparum. In other embodiments, the invention provides methods for treating malaria by administering a therapeutically effective amount of at least one of the pyridine derivatives of the invention to a patient in need thereof.

The invention provides methods for modulating the growth of parasites by administering an effective amount of at least one of the pyridine derivatives of the invention. The parasite is preferably a protozoan, more preferably Plasmodium falciparum. “Modulating the growth of parasites” includes inhibiting the growth of parasites; reducing the rate at which the parasites reproduce or grow (i.e., compared to untreated parasites); and/or killing the parasites. The growth of parasites can be modulated in vitro or in vivo.

The pyridine derivatives useful in the methods described herein are preferably compounds of formula (I), pharmaceutically acceptable salts thereof, or stereoisomers thereof:

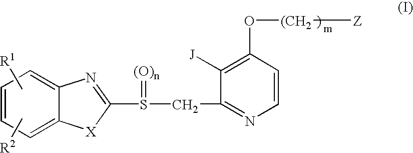

wherein R¹ and R² are each independently a hydrogen atom, a halogen atom, a lower alkyl, lower alkoxy, halogenated lower alkyl, lower alkoxycarbonyl or carboxyl group;

X is —O—, —S— or ═N—R³, wherein R³ is a hydrogen atom or a lower alkyl, phenyl, benzyl or lower alkoxycarbonyl group; and

Z is:

1. —O(CH₂)ₚ—O—R⁴
wherein p is an integer of 1 to 3 and R⁴ is hydrogen atom or a lower alkyl, aryl or aralkyl group,

2. —O—(CH₂)_{q}—R⁵
wherein q is an integer of 1 to 3 and R⁵ is a halogen atom or an alkoxycarbonyl, aryl or heteroaryl group,

3. —O—(CH₂)ᵣ—O—(CH₂)ₛ—O—R⁶
wherein r and s are each independently an integer of 1 to 5 and R⁶ is a hydrogen atom or a lower alkyl group,

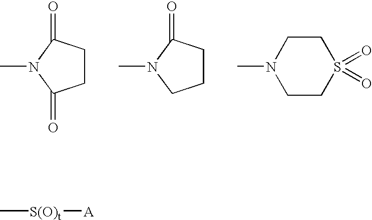

wherein t is an integer of 0 to 2, and A is a lower alkyl, alkoxycarbonylmethyl, pyridyl, furyl,

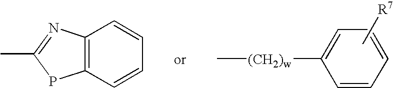

wherein B is —NH—, —O— or —S—, and w is an integer of 0 or 1;

8. —N(R⁸)—CH₂—C₆H₅
wherein R⁵ is an acetoxy or lower alkyl group;

9. —OR⁹

wherein R⁹ is a hydrogen atom, a lower alkyl or aryl group;

n is an integer of 0 to 2; m is an integer of 2 to 10, and J and K are each independently a hydrogen atom or a lower alkyl group, with the proviso that when Z is a group falling under the above category (9), then R⁹ is a lower alkyl group and m stands for an integer of 3 to 10, and pharmaceutically acceptable salts thereof.

The same definitions for R¹, R², X, n, J, K, Z and m are used throughout the specification that follows and in the appended claims.

Also disclosed are pharmaceutical compositions containing one or more of these compounds as the active ingredient(s) in a pharmaceutically acceptable carrier, adjuvant or vehicle.

In the definition of the compounds of formula (I), the lower alkyl group defined with respect to R¹, R², R³, R⁴, R⁶, R⁷, R⁸, A, J and K may be a straight-chain or branched alkyl group having 1 to 6 carbon atoms. Examples include methyl, ethyl, n-propyl, n-butyl, isopropyl, isobutyl, 1-methylpropyl, tert-butyl, n-pentyl, 1-ethylpropyl, isoamyl and n-hexyl groups, among which methyl and ethyl groups are most preferred.

The lower alkoxy group and the lower alkoxy moiety of the lower alkoxycarbonyl group defined above with respect to R¹ and R² may be an alkoxy group derived form the above lower alkyl group. Methoxy and ethoxy groups are most preferred.

The halogen atom defined above includes chlorine, bromine, iodine or fluorine. The aryl group defined above with respect to R⁴ and R⁵ may be phenyl, tolyl, xylyl, napthyl or the like, which may be substituted with a lower alkoxy or hydroxyl group, a halogen atom or the like.

Examples of the arylalkyl defined above with respect to R⁴ include benzyl and phenethyl groups.

Examples of the heteroaryl group defined above with respect to R⁵ include pyridyl and furyl groups.

In the definition of Z in formula (I), groups 1, 2, 3, 4, 5 and 9 are preferred; and group 9 is the most preferred. As for R¹ and R², hydrogens for both and then a combination of a lower alkyl (e.g., methyl) for R¹ and hydrogen for R² are preferred. X is preferably ═NR³, where R³ is hydrogen. A preferred value for n is 1. The preferred substituents for J and K are both hydrogen or where J is lower alkyl (e.g., methyl), and K is hydrogen, or when J is hydrogen and K is lower alkyl (e.g., methyl). Thus, J or K are independently preferably hydrogen or methyl, most preferably J is methyl and K is hydrogen.

A more preferred group of compounds falling within the scope of the compounds of formula (I) are compounds of formula (A), pharmaceutically acceptable salts thereof, or stereoisomers thereof:

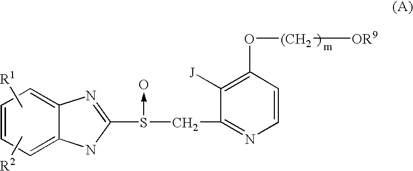

wherein R¹, R², J, m and R⁹ have the same meanings as defined above.

In formula (A), the preferred R¹ and R² substituents are both hydrogen, or R¹ is 5-lower alkoxy, 5-lower alkyl or 5-halogenated lower alkyl and R² is hydrogen. The preferred substituent for J is hydrogen or methyl; the preferred value for m is in the range of 3 to 10, the most preferred being 3; and the preferred R⁹ substituent is lower alkyl (e.g., methyl), or aryl. Among these possibilities for the compounds of formula (A), the preferred combination is when R¹ and R² are both hydrogen, J is methyl, m is 3 and R⁹ is methyl.

Another group of preferred compounds in formula (A) are combinations of the above substituents where both R¹ and R² are hydrogen, J is hydrogen, m is 3 and R⁹ is methyl.

Another group of preferred compounds falling within formula (A) is when both R¹ and R² are hydrogen, J is methyl, m is 2 and R⁹ is benzyl.

Another preferred group of compounds falling within the scope of formula (I) are compounds of formula (B), pharmaceutically acceptable salts thereof, or stereoisomers thereof:

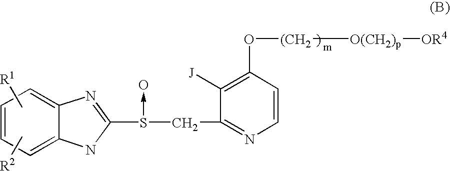

wherein R¹, R², J, p, m and R⁴ have the same meanings as given above.

In formula (B), the preferred substituents for R¹ and R² are both hydrogen; or when R¹ is 5-lower alkoxy, 5-lower alkyl or 5-halogenated lower alkyl, R² is hydrogen. The preferred value of m is 2 or 3; the preferred value for p is 2 or 3; and the preferred substituent for R⁴ is methyl or benzyl. Of the above possibilities for formula (B), the most preferred combination is where R¹ is 5-methyl, R² is hydrogen, J is methyl, m is 2, p is 2 and R⁴ is methyl.

In a most preferred embodiment, the compound of formula I is a compound of formula (C), a pharmaceutically acceptable salt thereof, or a stereoisomer thereof:

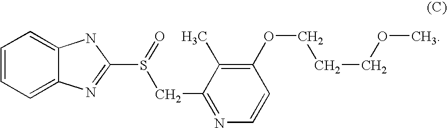

Preferably, the compound of formula (C) is a sodium salt, which is known as rabeprazole sodium or ACIPHEX® (Eisai Inc., Teaneck, N.J.).

Although the compounds of the invention may be present as a hydrate or as a stereoisomer, it is a matter of course that these hydrates and stereoisomers are also included in the scope of the invention. For example, the compound of formula (C) can be a compound of formula (D) or a pharmaceutically acceptable salt thereof (e.g., a sodium salt):

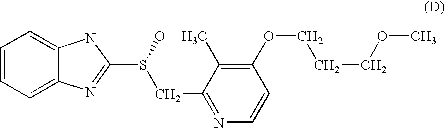

The compound of formula (D) is also known as R (+) rabeprazole.

Alternatively, the compound of formula (C) can be a compound of formula (E) or a pharmaceutically acceptable salt thereof (e.g., a sodium salt):

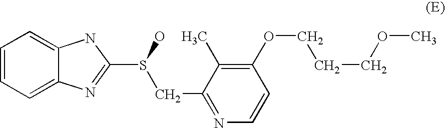

The compound of formula (E) is also known as S (−) rabeprazole.

As described above, the compounds of the invention can be administered as a pharmaceutically acceptable salt. Pharmaceutically acceptable salts are known in the art and include those of inorganic acids, such as hydrochloride, sulfate, hydrobromide, sulfate, and phosphate; those of organic acids, such as formate, acetate, maleate, tartrate, trifluoroacetate, methanesulfonate, benzenesulfonate and toluenesulfonate, and those of amino acids such as arginine, aspartic acid and glutamic acid. When certain substituents are selected, the compounds of the invention may form, for example, alkali metal salts, such as sodium or potassium salts; alkaline earth metal salts, such as calcium or magnesium salts; organic amine salts, such as a salt with trimethyl-amine, triethylamine, pyridine, picoline, dicyclohexylamine or N,N′-dibenzylethylene-diamine. One skilled in the art will recognize that the compounds of the invention can be made in the form of any of these or of any other pharmaceutically acceptable salt. For example, compounds represented by formula (I), wherein X is ═N—R³ and R³ is a hydrogen atom, or compounds represented by formula (I), wherein Z is a group falling under the category 7 and B is a group of —NH—, can be present as a metal salt, such as Na, K, Mg or Ca.

The pyridine derivatives of the invention can be prepared by processes that are known in the art and described, for example, in U.S. Pat. No. 5,045,552, the disclosure of which is incorporated by reference herein in its entirety. Rabeprazole sodium, a preferred pyridine derivative for use in the methods described herein, is commercially available as ACIPHEX® from Eisai Inc., Teaneck, N.J. Methods for preparing R (+) rabeprazole are described in WO 99/55157, the disclosure of which is incorporated by reference herein in its entirety. Methods for preparing S (−) rabeprazole are described in WO 99/55158, the disclosure of which is incorporated by reference herein in its entirety.

A therapeutically effective dosage regimen for treating the diseases described herein with the pyridine derivatives described herein is selected in accordance with a variety of factors, including the age, weight, sex, and medical condition of the patient, the severity of the disease, the route of administration, pharmacological considerations such as the activity, efficacy, pharmacokinetic and toxicology profiles of the particular pyridine derivative used, whether a drug delivery system is used and whether the pyridine derivative is administered as part of a drug combination.

The pyridine derivatives described herein may be administered in amounts of about 0.01 to about 200 mg per day, preferably about 0.05 to about 50 mg per day, more preferably about 0.1 to about 40 mg per day, still more preferably about 10 to about 30 mg per day, most preferably about 20 mg per day. The compounds and/or compositions may be administered once a day or in divided doses, for example from 2 to 4 times a day, most preferably once per day. One skilled in the art will recognize that when the pyridine derivatives of the invention are administered to infants or children, the dose may be smaller than the dose administered to adults, and that the dose can be dependent upon the size and weight of the patient.

In the methods for treating and preventing refractory ulcers, for treating and preventing Zollinger-Ellison syndrome, and for treating and preventing idiopathic gastric acid hypersecretion described herein, the patient may be administered at least one of the pyridine derivatives of the invention in amounts of about 1 to about 800 mg per day, preferably about 10 to about 300 mg per day, more preferably about 20 to about 200 mg per day, still more preferably about 40 to about 150 mg per day, most preferably about 60 to about 120 mg per day.

In preferred embodiments of the methods described herein, rabeprazole sodium, which is commercially available as ACIPHEX® (Eisai Inc., Teaneck, N.J.), is administered as a delayed-release, enteric-coated tablet containing 20 milligrams rabeprazole sodium. The tablets can be administered one to about four times a day. In preferred embodiments, one 20 milligram ACIPHEX® tablet is administered once a day for the methods described herein. One skilled in the art will appreciate that when rabeprazole sodium is administered to infants or children, the dose may be smaller than the dose that is administered to adults.

The pyridine derivatives of the invention can be administered orally, topically, parenterally, by inhalation (nasal or oral), or rectally in dosage unit formulations containing conventional nontoxic pharmaceutically acceptable carriers, adjuvants, and vehicles as desired. The term parenteral as used herein includes subcutaneous, intravenous, intramuscular, intrasternal injection, or infusion techniques. Preferably, the pyridine derivatives of the invention are orally administered as tablets.

Injectable preparations, for example, sterile injectable aqueous or oleaginous suspensions may be formulated according to the known art using suitable dispersing or wetting agents, suspending agents (e.g., methylcellulose, Polysorbate 80, hydroxyethylcellulose, acacia, powdered tragacanth, sodium carboxymethylcellulose, polyoxyethylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), preservatives and/or stabilizers. The sterile injectable preparation may also be a sterile injectable solution or suspension in a nontoxic parenterally acceptable diluent or solvent, for example, as a solution in 1,3-butanediol. Among the acceptable vehicles and solvents that may be used are water, Ringer's solution, and isotonic sodium chloride solution. In addition, sterile, fixed oils are conventionally used as a solvent or suspending medium. For this purpose any bland fixed oil may be used including synthetic mono- or diglycerides, in addition, fatty acids such as oleic acid find use in the preparation of injectables. The preparations can be lyophilized by methods known in the art.

Solid dosage forms for oral administration may include capsules, tablets, sublingual tablets, powders, granules and gels; most preferably tablets. The solid dosage form may be a solid microencapsulated dosage, such as a microencapsulated powder, microencapsulated granules or a microencapsulated gel. A solid dosage form for oral administration can be prepared by mixing an active principle with filler and, if necessary, binder, disintegrating agent, lubricant, coloring agent, corrigent or the like and converting the obtained mixture into a tablet, coated tablet, granule, powder or capsule. Examples of the filler include lactose, corn starch, sucrose, glucose, sorbitol, crystalline cellulose and silicon dioxide, while those of the binder include polyvinyl alcohol, polyvinyl ether, ethylcellulose, methylcellulose, acacia, tragacanth, gelatin, shellac, hydroxypropylcellulose, hydroxypropylstarch and polyvinylpyrrolidone. Examples of the disintegrating agent include starch, agar, gelatin powder, crystalline cellulose, calcium carbonate, sodium hydrogencarbonate, calcium citrate, dextrin and pectin, while those of the lubricant include magnesium stearate, talc, polyethylene glycol, silica and hardened vegetable oils. The coloring agent may be any one which is permitted to be added to drugs. Examples of the corrigent include cacao powder, mentha herb, aromatic powder, mentha oil, borneol and powdered cinnamon bark. The tablets and granules may be, if necessary, coated with sugar, gelatin or the like. Preferably, the tablets have an enteric coating.

In addition to the active ingredient, the tablets preferably comprise mannitol, hydroxypropyl cellulose, magnesium oxide, low-substituted hydroxypropyl cellulose, magnesium stearate, ethylcellulose, hydroxypropyl methylcellulose phthalate, diacetylated monoglycerides, talc, titanium dioxide, carnauba wax and a coloring agent, such as ferric oxide.

In other embodiments, the solid dosage form can be packaged as granules or a powder in a pharmaceutically acceptable carrier, where the granules or powder are removed from the packaging and sprinkled on food or mixed with a liquid, such as water or juice. In this embodiment, the active compound can be mixed with flavoring or sweetening agents. The packaging material can be plastic, MYLAR® (DuPont), coated paper, or any material that prevents water or moisture from reaching the granules and/or powder.

Liquid dosage forms for oral administration can include pharmaceutically acceptable emulsions, solutions, suspensions, and syrups containing inert diluents commonly used in the art, such as water. The liquid dosage form may be a microencapsulated liquid, including microencapsulated emulsions, microencapsulated solutions, microencapsulated suspensions and microencapsulated syrups. Such compositions can also comprise adjuvants, such as wetting agents, emulsifying and suspending agents, and sweetening, flavoring, and perfuming agents.

For administration by oral or nasal inhalation, the compositions of the invention can be delivered from an insufflator, a nebulizer or a pressured pack or other convenient mode of delivering an aerosol spray. Pressurized packs can include a suitable propellant. Alternatively, for administration by oral or nasal inhalation, the compositions can be administered in the form of a dry powder composition or in the form of a liquid spray.

Suppositories for rectal administration can be prepared by mixing one or more pyridine derivatives of the invention with suitable nonirritating excipients, such as cocoa butter and/or polyethylene glycols, that are solid at room temperature and that melt at body temperature.

For topical administration to the epidermis, the pyridine derivatives of the invention can be formulated as ointments, creams or lotions, or as the active ingredient of a transdermal patch. Ointments and creams may, for example, be formulated with an aqueous or oily base with the addition of suitable thickening and/or gelling agents. Lotions may be formulated with an aqueous or oily base and can also generally contain one or more emulsifying agents, stabilizing agents, dispersing agents, suspending agents, thickening agents, and/or coloring agents. The pyridine derivatives can also be administered via iontophoresis.

While the pyridine derivatives of the invention can be administered as the sole active pharmaceutical agent in the methods described herein, they can also be used in combination with one or more compounds which are known to be therapeutically effective against the specific disease that one is targeting for treatment.

Each of the patents and publications cited herein are incorporated by reference herein in their entirety.

It will be apparent to one skilled in the art that various modifications can be made to the invention without departing from the spirit or scope of the appended claims.

What is claimed is:

1. A method for treating or preventing symptomatic gastroesophageal reflux disease in a patient in need thereof comprising administering a therapeutically effective amount of a compound of formula (C), a pharmaceutically acceptable salt thereof, or a stereoisomer thereof:

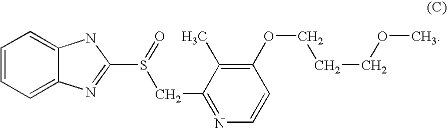

2. The method of claim 1, wherein the compound of formula (C) is rabeprazole sodium or a stereoisomer thereof.

3. The method of claim 1, wherein the compound of formula (C) is a compound of formula (D) or a pharmaceutically acceptable salt thereof:

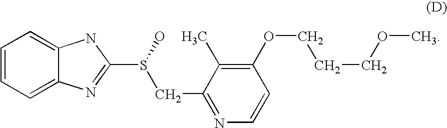

4. The method of claim 3, wherein the compound of formula (D) is a sodium salt.

5. The method of claim 1, wherein the compound of formula (C) is a compound of formula (E) or a pharmaceutically acceptable salt thereof:

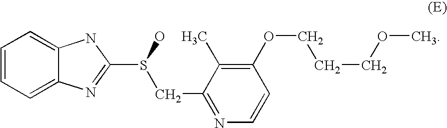

6. The method of claim 5, wherein the compound of formula (E) is a sodium salt.

7. The method of claim 1, wherein the compound of formula (C), the pharmaceutically acceptable salt thereof or the stereoisomer thereof is orally administered.

8. The method of claim 7, wherein the compound of formula (C), the pharmaceutically acceptable salt thereof or the stereoisomer thereof is orally administered in the form of a tablet.

9. The method of claim 1, wherein the compound of formula (C), the pharmaceutically acceptable salt thereof or the stereoisomer thereof is administered in an amount of about 20 milligrams per day.

10. A method for treating or preventing symptomatic gastroesophageal reflux disease in a patient in need thereof comprising administering a therapeutically effective amount of a compound of formula (I), a pharmaceutically acceptable salt thereof, or a stereoisomer thereof: 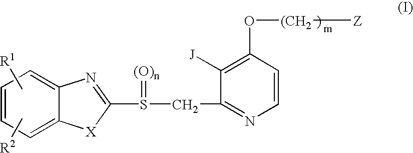
wherein R¹ and R² are each independently a hydrogen atom, a halogen atom, a lower alkyl, lower alkoxy, halogenated lower alkyl, lower alkoxycarbonyl or carboxyl group;
X is —O—, —S— or ═N—R³, wherein R³ is a hydrogen atom or a lower alkyl, phenyl, benzyl or lower alkoxycarbonyl group; and
Z is:
(i) —O(CH₂)ₚ—O—R⁴
wherein p is an integer of 1 to 3 and R⁴ is hydrogen atom or a lower alkyl, aryl or aralkyl group,
(ii) —O—(CH₂)_{q}—R⁵ wherein q is an integer of 1 to 3 and R⁵ is a halogen atom or an alkoxycarbonyl, aryl or heteroaryl group,
(iii) —O—(CH₂)ᵣ—O—(CH₂)ₛ—O—R⁶
wherein r and s are each independently an integer of 1 to 5 and R⁶ is a hydrogen atom or a lower alkyl group, 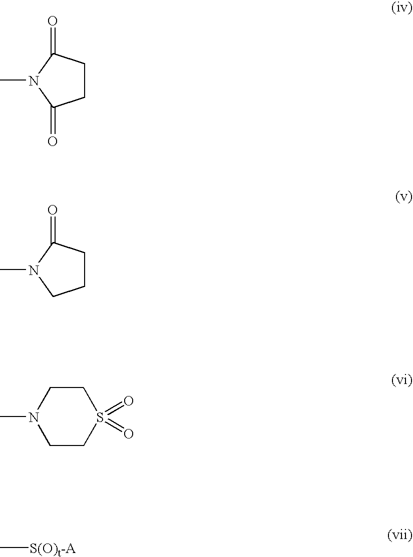
wherein t is an integer of 0 to 2, and A is a lower alkyl, alkoxycarbonylmethyl, pyridyl, furyl, 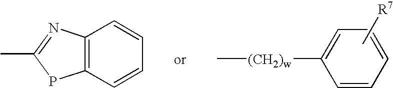
wherein B is —NH—, —O— or —S—, and w is an integer of 0 or 1;
(viii) —N(R⁸)—CH₂—C₆H₅
wherein R⁸ is an acetoxy or lower alkyl group; or
(ix) —OR⁹
wherein R⁹ is a hydrogen atom, a lower alkyl or aryl group;
n is an integer of 0 to 2; m is an integer of 2 to 10, and J and K are each independently a hydrogen atom or a lower alkyl group, with the proviso that when Z is —OR⁹, then R⁹ is a lower alkyl group and m is an integer of 3 to 10.

11. The method of claim 10, wherein the compound of formula (I) is a compound of formula (A), a pharmaceutically acceptable salt thereof, or a stereoisomer thereof: 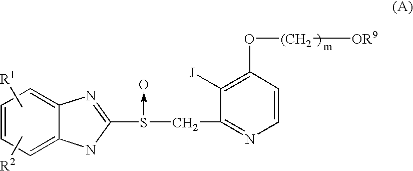
wherein R¹, R², J, m and R⁹ are as defined above.

12. The method of claim 5, wherein the compound of formula (I) is a compound of formula (B), a pharmaceutically acceptable salt thereof, or a stereoisomer thereof: 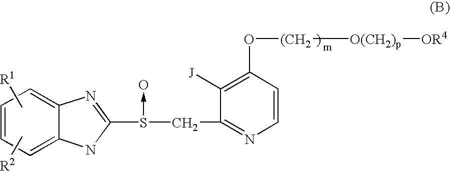
wherein R¹, R², J, p, m and R⁴ are as defined above.

13. The method of claim 12, wherein the compound of formula (I) is a compound of formula (C), a pharmaceutically acceptable salt thereof, or a stereoisomer thereof:

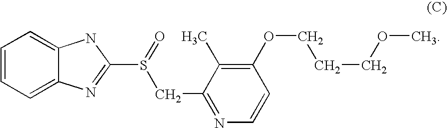

14. The method of claim 13, wherein the compound of formula (C) is rabeprazole sodium or a stereoisomer thereof.

15. The method of claim 13, wherein the compound of formula (C) is R (+) rabeprazole or a pharmaceutically acceptable salt thereof.

16. The method of claim 13, wherein the compound of formula (C) is S (−) rabeprazole or a pharmaceutically acceptable salt thereof.

17. The method of claim 10, wherein the compound of formula (I), the pharmaceutically acceptable salt thereof, or the stereoisomer thereof is administered orally.

18. The method of claim 12, wherein the compound of formula (I), the pharmaceutically acceptable salt thereof, or the stereoisomer thereof is orally administered as a solid dosage form or a liquid dosage form.

19. The method of claim 18, wherein the solid dosage form is a capsule, a tablet, a sublingual tablet, a powder, a granule or a gel.

20. The method of claim 10, wherein the compound of formula (I), the pharmaceutically acceptable salt thereof, or the stereoisomer thereof is administered in an amount of about 0.01 to about 200 milligrams per day.

21. The method of claim 21, wherein the compound of formula (I), the pharmaceutically acceptable salt thereof, or the stereoisomer thereof is administered in an amount of about 0.1 to about 40 milligrams per day.

22. The method of claim 21, wherein the compound of formula (I), the pharmaceutically acceptable salt thereof, or the stereoisomer thereof is administered in an amount of about 10 to about 30 milligrams per day.
